(19) United States
(12) Patent Application Publication
(10) Pub. No.: US 2010/0094416 A1
(43) Pub. Date: Apr. 15, 2010

(54) SOFT TISSUE IMPLANT

(76) Inventors: Frank Maguire, Salt Lake City, UT (US); Ajay Padsalgikar, Victoria (AU); Leroy Young, Wildwood, MO (US); Mark Jewell, Eugene, OR (US)

Correspondence Address: SCHWEGMAN, LUNDBERG & WOESSNER, P.A., P.O. BOX 2938, MINNEAPOLIS, MN, 55402, US

(21) Appl. No.: 12/444,676

(22) PCT Filed: Oct. 10, 2006
(86) PCT No.: PCT/AU06/01488

§ 371 Date: Dec. 8, 2009

(51) Int. Cl. A61F 2/12

(52) U.S. Cl. 623/8

(57) ABSTRACT

A soft tissue implant comprises a shell filled with a fluid biomaterial that is capable of curing in situ. A method of implanting a soft tissue implant includes: creating a pocket in soft tissue thereby defining the location of the implant; inserting an unfilled shell into the pocket; filling the shell with a biomaterial to a predetermined volume; and curing the biomaterial in situ.

The present invention relates to soft tissue implants and a method of implanting soft tissue implants.

### BACKGROUND

Implants for the human body for the purpose of cosmetic or reconstructive surgery have been used for many years with varying degrees of success. The most popularly implanted implants are breast implants for augmentation mammoplasty. Silicone gel encased in a silicone elastomer shell forms one of the most widely used type of breast implant.

The surgical operation using silicone gel breast implants involves placing the implant through an incision of typically 5 cm or more in length. The device is squeezed in this procedure and the stress imparted to the implant by this procedure has been shown to affect the strength of the shell and to be a significant factor contributing to the failure of these types of implants. Additionally, this is a major operation requiring the recipient of the implant to be placed under general anaesthesia.

Growing health concerns observed over time with silicone gel implants have caused severe restrictions to be imposed on the use of implants by regulatory authorities in certain jurisdictions. In particular, gel filled implants have a propensity to leach out of the shell and potentially impact the health of the recipient.

Another type of widely used implant is the saline filled implant which was brought about in order to address the problem of leaching. However, saline implants fail to replicate the consistency and texture of natural breast tissue and tend to exhibit ‘fold flow’, or rippling, at the implant edges causing a generally unnatural feel and shape.

The problems associated above with breast implants, which follow through to tissue implants in general, are addressed with the implant and method of implanting of the present invention.

### SUMMARY OF INVENTION

In accordance with the present invention there is provided a soft tissue implant comprising a shell filled with a liquid biomaterial that is capable of curing in situ.

In accordance with the present invention there is further provided a method of implanting a soft tissue implant including:
creating a pocket in soft tissue thereby defining the location of the implant;
inserting an unfilled shell into the pocket;
filling the shell with a biomaterial to a predetermined volume; and
curing the biomaterial in situ.

### BRIEF DESCRIPTION OF THE DRAWINGS

The present invention is described further by way of example with reference to the accompanying drawings by which:

FIG. 1 is a schematic diagram illustrating a method of implanting a soft tissue implant in accordance with an embodiment of the present invention;

FIG. 2(a) is a side schematic view of an embodiment of a soft tissue implant being located in soft tissue;

FIG. 2(b) illustrates the soft tissue implant of FIG. 2(a) being filled with fill material; and

FIG. 3 is a side schematic partial view of another embodiment of a soft tissue implant;

### DETAILED DESCRIPTION OF PREFERRED EMBODIMENT

A soft tissue implant 10 and a minimally invasive method of implanting a soft tissue implant 10 is schematically illustrated in the drawings. The implant can be used to reconstruct or alter any suitable part of a mammal, preferably a human including breasts, buttocks, legs, arms, and facial areas such as cheeks and the chin. In situ, that is, implanted in soft tissue, the implant comprises an elastomeric shell 12 containing a biomaterial having consistency and texture comparative to the natural consistency of the body area to be reconstructed.

In the following description and drawings, specific reference is made to breast implants and a method of implanting breast implants as an example of the present invention, although it is understood that the implant and method can apply equally to any soft tissue implant.

The term soft tissue includes all connective tissue aside from bones and teeth.

The biomaterial used to fill the shell 12 is a silicon-containing biomaterial and, in the preferred embodiment is a biostable silicon-containing gel that is capable of being cured in situ in the soft tissue in which the implant is implanted. Biomaterial in the form of silicon-containing gel must be cured in order to achieve biostability and correct consistency. In known techniques silicon-containing gel is cured inside the shell prior to implanting. Saline filled implants do not require curing.

The present technique advantageously uses an uncured fluid silicon-containing gel that can be delivered to the implant shell after the shell is inserted into a human body, and then cured in situ. Accordingly, only a minimally invasive surgical procedure is required where a small incision is made to the tissue just large enough to allow the empty shell and liquid biomaterial to pass therethrough.

The term “fluid” means that the biomaterial is in a state capable of being delivered into the shell. The term includes liquid and other flowable biomaterials such as viscous biomaterials and partially cured biomaterials.

The term “biomaterial” refers to a material which is used in situations where it comes into contact with cells and/or bodily fluids of living mammals such as humans and animals. The biomaterial is preferably biostable which means that it is stable when it comes into contact with cells and/or bodily fluids of living mammals. It is also preferable for the biomaterial to have low exotherm properties limited to 4° C. so as to avoid heating and damaging the surrounding tissue during curing and low extractables of preferably less than 35%.

The term “extractables” refers to the unreacted portion of the biomaterial which is generally fluid and free to migrate out of the biomaterial at body temperature of 35° C. and more specifically, refers to the unreacted fluid portion of a biomaterial which is extracted by organic solvents at temperatures in the range from 20° C. to 40° C.

The biomaterial is preferably in the form of a gel which replicates the consistency and texture of natural tissue. The gel may be a chemical or physical gel, preferably a chemical gel. Representative examples of biostable silicon-containing gels which are fluid and capable of curing in situ are as follows:
1. Isocyanate terminated prepolymer (with varying percentage of NCO) which is synthesised from 4, 4′-diphenylmethane diisocyanate (MDI) and polydimethylsiloxane (Mw 1000/2000). The required amount of higher molecular weight synthesised T-Triol (˜40000-70000) is added, mixed mechanically with few drops of metal catalyst for about 5 minutes and kept aside in room temperature for ˜15 min for gelling.
2. Hydroxyethoxypropyl terminated 3.55%-(methylmethacryloxypropyl methyl siloxane) (dimethyl siloxane) copolymer (Mw˜50000-100000) and a photoinitator (Irgacure 2022) diluted in synthesised silicone diacrylate are mixed mechanically for 5 minutes and placed under an ultra high intensity UV-A lamp (365 nm) for ˜5 min to cure.
3. High molecular weight isocyanate terminated (propyl methyl siloxane) (dimethyl siloxane) copolymer and diol (Mw 100-2000) are mixed mechanically with few drops of metal catalyst for about 5 minutes and kept aside at room temperature for ˜15min for gelling.
4. Hydroxy terminated siloxane diols and triols with vinyl groups in the polymer chain are synthesised, reacted with prepolymer synthesised from 4, 4′-diphenylmethane diisocyanate (MDI) and polydimethylsiloxane (Mw 1000/2000) with varying percentages of NCO and then subjected to UV curing.
5. A mixture of α, ω-bis (γ-aminopropyl) polydimethylsiloxane, isocyanoethyl methacrylate with 0.6% photoinitiator and reactive diluent (ethyl methacrylate, hydroxylmethyl methacrylate, butyl acrylate, acrylic acid, methyl methacrylate and vinyl pyridine) is poured into an aluminium dish. The mixture is irradiated from one side under a nitrogen atmosphere using a bank of 20-W mercury lamps (λ=365 nm) as the irradiation source. An irradiation time of 30 min is found to completely cure the mixture.
6. Hydroxyethoxypropyl terminated epoxyglycidylethersiloxane) copolymer (Mw˜50000-100000) and α, ω-bis (γ-aminopropyl) polydimethylsiloxane are mixed mechanically for 5 minutes and kept aside in room temperature for ˜15 min for gelling.
7. Epoxyglycidylethersiloxane terminated (methylmethacryloxypropyl methyl siloxane) (dimethyl siloxane) copolymer (Mw˜50000-100000) and photoinitator (Irgacure 2022) diluted in synthesised silicone diacrylate are mixed mechanically for 5 minutes and placed under an ultra high intensity UV-A lamp (365 nm) for ˜5 min to cure.
8. High molecular weight tri-vinyl terminated siloxane copolymer (Mw 50000-10000) and a photoinitator (Irgacure 2022) diluted in synthesised silicone diacrylate are mixed mechanically for 5 minutes and placed under an ultra high intensity UV-A lamp (365 nm) for ˜5 min to cure.

Preferably, the biostable silicon-containing gel is of the type disclosed in W02006/034547 and AU 2006902072, the entire contents of which are incorporated herein by reference.

Shell 12 is an elastomeric shell made from polyurethane, polyurethane urea or polycarbonate such as silicon-containing polyurethane, polyurethane urea or polycarbonate; or silicon manufactured by a suitable means including dipping, blow moulding, fusing opposite shell sides, or the like. Examples of silicon-containing polyurethanes, polyurethane ureas or polycarbonates include those disclosed in WO92/00338, WO98/13405, WO98/54242, WO99/03863 and WO00/64971, the entire contents of which are incorporated herein by reference. In the preferred embodiment the shell is formed from an Elast-Eon® polymer or is a silicon shell lined with an Elast-Eon® polymer. The shell is either symmetrical or profiled to a specific implant shape. The shell may have a smooth or textured external surface. A textured surface reduces the amount of scar tissue forming around the implant.

The shape of the implant can be determined by the use of a shell such as Elast-Eon with a modulus capable of providing adequate elongation to be appropriate as a soft tissue implant but with an elongation limit below the ability of the biomaterial fill to distort the desired shape of the implant.

As illustrated in the Figures the shell 12 includes an aperture 14 through which a fill delivery tool, in this embodiment a narrow bore flexible stylet 20, is inserted in order to fill the implant shell with silicon-containing gel.

In its uncured state the silicon-containing gel is a liquid monomer having a high or low viscosity and is therefore deliverable or injectable through the stylet. Upon curing the silicon-containing gel becomes polymeric, namely a viscoelastic gel.

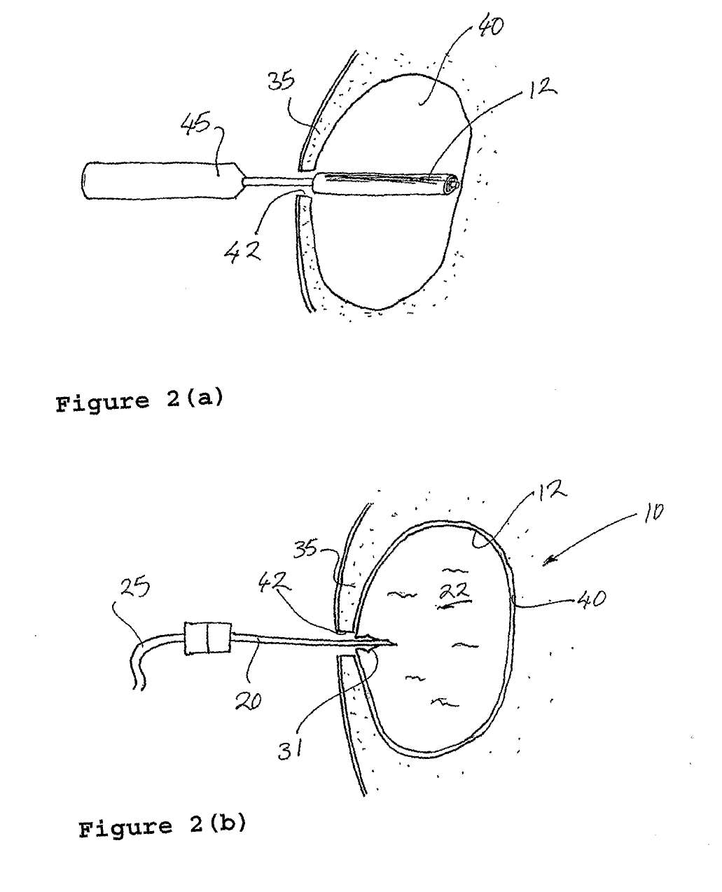

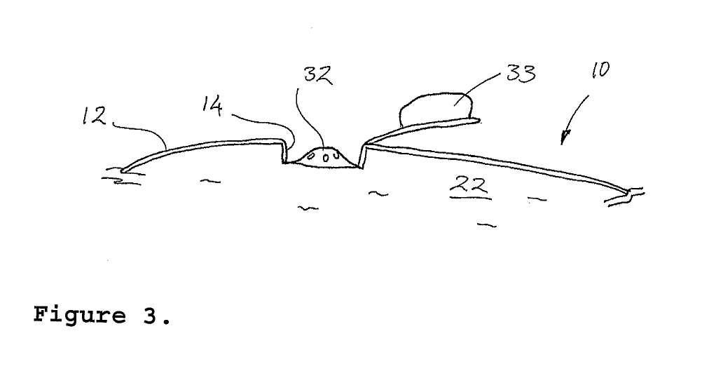

A valve 30, and in particular a check valve or one-way valve, may be incorporated at the aperture to prevent the liquid silicon-containing gel leaking before it has fully cured. FIG. 2(b) illustrates one type of valve that may be used—a duckbill valve 31. FIG. 3 illustrates an alternative embodiment of the shell using a diaphragm valve 32 with an external plug 33. These valves allow sealed access to the fill stylet. Once filling is completed the stylet is removed and the valves prevent the liquid gel from leaking back out of the shell.

The plug 33 may be used on its own without a diaphragm valve such that the surgeon must quickly plug the aperture after filling before leakage can occur. An external plug 33, such as the plug illustrated, or an internal plug may be used.

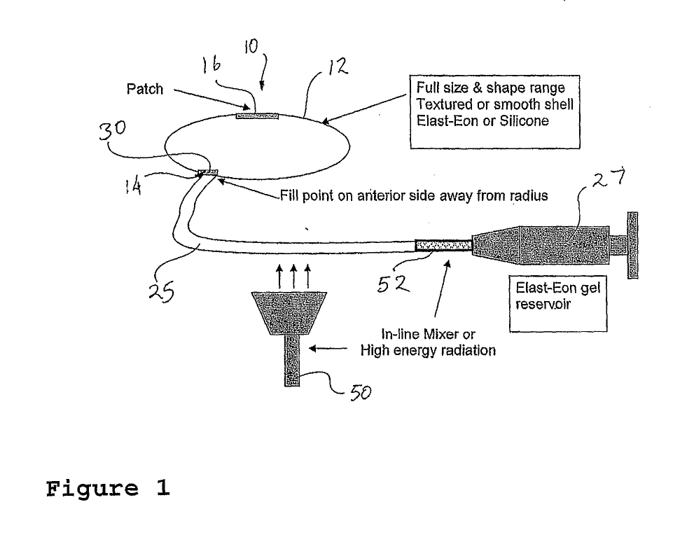

Other types of valves that may be incorporated into the shell include reed valves, leaf valves, cross slit valves or the like. The valves are integrally formed with the elastomeric shell during the manufacturing process. FIG. 1 illustrates a patch 16 defining the material join formed during manufacturing. In some implant applications, and depending on the shape of the shell, it may be suitable to locate the aperture 14 at the material join.

An alternative to using a valve 30 is using no valve. In this case excess liquid gel is allowed to flow out of the aperture 14. As the silicon-containing gel cures outflow ceases and the excess hardened thread of biomaterial gel is trimmed at the aperture.

The surgical procedure for implanting the soft tissue implant 10 requires the creation of a pocket 40 into which the empty shell 12 is placed. Depending on the nature of the implant and the area where it is to be located in a patient, the patient may be anaesthetised locally or generally.

To create the pocket 40 a small hole 42 is incised through the skin and tissue 35 at the access point using standard surgical instruments. For breast implant procedures the hole need only be approximately 1 cm in diameter/length. Other procedures may require a smaller or greater hole that is largely determined by the size of the empty shell to be inserted. On average it is considered that the hole size will range between 0.5-5 cm.

Soft tissue under the skin is removed through the hole 42 by surgical suction and/or is displaced by a balloon dissector, or the like, to create a space for the pocket. Pocket 40 is enlarged, typically by inflating a balloon dissector, to correct dimensions corresponding to the size of the implant to be implanted. Correct pocket size is verified by taking measurements of the pocket and/or visually inspecting the pocket using a fibre optic retractor or endoscope. A trial implant using a sizer and filling it with air may also be used.

Once the pocket dimensions are verified, the empty shell 12 is inserted into the pocket in a compact state. This means the pocket is inserted folded or rolled. In this embodiment the shell is rolled, or wrapped, onto the end of a hollow and flexible insertion stylet, such as a trocar. As illustrated in FIG. 2(a) an insertion stylet 45 with shell 12 wrapped around one end is inserted through hole 42 and into pocket 40.

At this point the insertion stylet is positioned at one end of the pocket and carefully rotated towards the other end of the pocket to unroll the shell off the stylet. A lubricant may be used to ease shell insertion which is carried out in a manner to cause negligible stress to the shell thereby maintaining shell integrity and strength. Unraveled, the shell 12 lies substantially flat within the pocket. Aperture 14, generally including valve 30, locates in alignment with hole 42. Insertion stylet 45 is removed.

Fill stylet 20 is then inserted through valve 30 and liquid biomaterial is delivered from a reservoir through tubing to the stylet and into the shell 12. FIG. 2(b) illustrates fill stylet 20 protruding through valve 31 and filling shell 12 with biomaterial 22. The biomaterial may be delivered by intravenous style under head pressure from a bag suspended above the patient. Alternatively, the biomaterial may be manually injected from a syringe 27, as illustrated in FIG. 1, and through a tube 25 to which the fill stylet 20 is attached. Yet another form of delivery may comprise the biomaterial to be delivered by way of a mechanical pump, such as a peristaltic pump.

Viscous biomaterial fills the shell to a predetermined volume determined for the particular size and shape of the shell. Under filling and overfilling is undesirable. Under filling may result in rippling and a generally unnatural shape including problems with positioning, while overfilling can lead to pleating (scalloping) and shape distortion.

The biomaterial is cured in situ. Curing is either initiated before the biomaterial enters the shell, that is during delivery, with curing completing inside the shell, or the biomaterial cures entirely within the shell.

External curing techniques can be used where curing is initiated outside the shell, and therefore outside the body. Such techniques include radiating the passing biomaterial with infra red light, ultra violet light, ultrasonic energy, or the like, as the biomaterial is delivered into the shell. FIG. 1 illustrates a high energy radiation device 50 radiating tube 25 containing biomaterial to initiate curing which is completed some minutes later when the biomaterial is in the shell.

Instead of initiating curing during delivery, a high energy radiation device may alternatively be placed inside hole 42 and adjacent the implant after biomaterial filling is completed.

Alternatively, curing may be initiated chemically by mixing the biomaterial in the form of its constituent monomers or as a pre-polymer with a curative cross linking agent in line with tube delivery. It will be appreciated that the biomaterial may contain other conventional additives used for polymer processing such as catalysts and initiators which also assist curing. FIG. 1 illustrates a mixer 52 located in line with tube 25 which connects between syringe 27 and fill stylet 20. As discussed, a catalyst may be introduced into the chemical mixture to accelerate curing.

In yet another alternative the biomaterial can be cured using the patient's own body temperature alone without the need for external assistance. It is also understood that in the above described external and chemical curing techniques, the patient's body temperature can also assist in accelerating curing.

It is further envisaged that during curing the implant may be externally supported by a device, such as a brassiere, which can mould the ultimate shape of the implant.

The above examples are illustrative of techniques where curing is initiated before closure of hole 42. Curing may also occur after closure. This may be carried out by: relying solely on the body temperature of the patient; or applying high energy radiation in the form of heat, ultrasound or light radiation, externally over the implant area.

For minimally invasive surgery it is envisaged that the time for curing will be under 20 minutes to cause as little as possible inconvenience and discomfort for the patient, especially if local anesthetic is used.

The shell may additionally carry radio-opaque markings to verify correct positioning within a patient after implanting and before and after the pocket is closed.

Pocket closure is carried out by sealing hole 42 using standard means including stitching or medical adhesive.

The present soft tissue implant and method of implanting the implant bring about major advantages in the field of cosmetic and reconstructive surgery. The minimally invasive nature of the surgery is less traumatic to the patient and allows the procedure to be carried out more efficiently and quickly than known techniques.

An additional advantage of the present implant is that the viscosity of the cured biomaterial fill can be controlled and may be varied to suit the consistency of the area to be implanted. For example, an implant for the facial area would be cured firmer than for a breast implant.

The implant can be safely used with confidence that leaching will not occur. Furthermore, it is envisaged that the present implant will require replacing far less regularly than known implants. Known implants require replacement on an average of every 5 years. The present soft tissue implant is envisaged to only require replacement once every 20 years.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

It will be understood to persons skilled in the art of the invention that many modifications may be made without departing from the spirit and scope of the invention.

1. A soft tissue implant comprising a shell filled with a fluid biomaterial that is capable of curing in situ.

2. The soft tissue implant claimed in claim 1 wherein the biomaterial is biostable.

3. The soft tissue implant claimed in claim 1 wherein the biomaterial is silicon-containing.

4. The soft tissue implant claimed in claim 3 wherein the biomaterial is a silicon-containing gel.

5. The soft tissue implant claimed in claim 4 wherein the exothermic properties of the gel are limited to a maximum temperature difference of +4° C.

6. The soft tissue implant claimed in claim 4 wherein the gel contains extractables of less than 35%.

7. The soft tissue implant claimed in claim 1 wherein the shell contains an aperture through which biomaterial is filled.

8. The soft tissue implant claimed in claim 7 wherein the aperture is defined by a check valve.

9. The soft tissue implant claimed in claim 8 wherein the valve is a diaphragm valve, a duckbill valve, a reed valve, a leaf valve, a cross slit valve or a plug valve.

10. The soft tissue implant claimed in claim 1 wherein the shell is made of an elastomeric material.

11. The soft tissue implant claimed in claim 10 wherein the elastomeric material is a polyurethane, polyurethane urea, polycarbonate and/or silicon.

12. The soft tissue implant claimed in claim 10 wherein the material is an ElastEon® polymer.

13. The soft tissue implant claimed in claim 1 wherein the shell contains radio-opaque markings for verification of location.

14. The soft tissue implant claimed in claim 10 wherein the elastomeric shell has a modulus of elongation such that the shell expands without distorting upon filling with biomaterial.

15. A method of implanting a soft tissue implant including:
creating a pocket in soft tissue thereby defining the location of the implant;
inserting an unfilled shell into the pocket;
filling the shell with a biomaterial to a predetermined volume; and
curing the biomaterial in situ.

16. The method claimed in claim 15 including closing the pocket before or after curing.

17. The method claimed in claim 15 including initiating curing of the biomaterial during filling such that curing is completed in situ.

18. The method claimed in claim 15 including curing the biomaterial after filling the shell.

19. The method claimed in claim 15 including curing the biomaterial externally.

20. The method claimed in claim 19 including using infra-red radiation, ultraviolet radiation or ultrasonic energy to externally cure the biomaterial.

21. The method claimed in claim 15 including curing the biomaterial chemically.

22. The method claimed in claim 21 including mixing the constituent monomers or a pre-polymer of the biomaterial and a curative cross linking agent and filling the shell with the biomaterial.

23. The method claimed in claim 22 including adding a catalyst and/or initiator to the biomaterial mixture.

24. The method claimed in claim 15 including supporting the implant during curing to define the shape of the implant.

25. The method claimed in claim 24 including supporting the implant with a brassiere.

26. The method claimed in claim 15 including filling the shell through a hole located on the shell.

27. The method claimed in claim 26 including filling the shell through a valve provided at the hole.

28. The method claimed in claim 15 wherein the empty shell is provided rolled onto an end of a stylet, inserting the stylet into the pocket and unrolling the empty shell off the stylet and into position.

29. The method claimed in claim 15 including delivering fluid biomaterial to the empty shell through a tube under pressure.

30. The method claimed in claim 29 including delivering the biomaterial to the empty shell under gravity.

31. The method claimed in claim 29 including injecting the biomaterial into the empty shell using a syringe.

32. The method claimed in claim 29 including pumping the biomaterial into the empty shell using a pump.

33. The method claimed in claim 15 including creating a pocket by first making an incision through the soft tissue and then clearing an area in the soft tissue to form a pocket.

34. The method claimed in claim 33 including making an incision between 0.5 cm and 5 cm in length.

35. The method claimed in claim 15 including confirming pocket size before shell insertion by measurement, visual means and/or trialing a sizer.
